(19) United States
(12) Patent Application Publication
(10) Pub. No.: US 2024/0331861 A1
(43) Pub. Date: Oct. 3, 2024

(54) METHOD AND SYSTEM FOR PREDICTING HISTOPATHOLOGY OF LESIONS

(71) Applicant: KONINKLIJKE PHILIPS N.V., EINDHOVEN (NL)

(72) Inventor: ABHIVYAKTI SAWARKAR, SWANSEA, MA (US)

(21) Appl. No.: 18/293,966

(22) PCT Filed: Jul. 21, 2022
(86) PCT No.: PCT/EP2022/070519

§ 371 Date: Jan. 31, 2024

Related U.S. Application Data
(60) Provisional application No. 63/228,180, filed on Aug. 2, 2021.

(51) Int. Cl. G16H 50/20; G06T 7/00; G16H 10/60; G16H 20/00; G16H 30/40; G16H 50/70

(52) U.S. Cl. CPC G16H 50/20; G06T 7/0014; G16H 10/60; G16H 20/00; G16H 30/40; G16H 50/70; G06T 2207/20081; G06T 2207/20084; G06T 2207/30096

(57) ABSTRACT

A system and method are provided for determining histological nature and medical treatment for lesions seen on medical images of a patient. The method includes detecting a lesion in a medical image; extracting image findings from a radiology report describing the lesion using NLP; retrieving demographic and clinical data of the patient from a database; identifying similar patients based on the demographic and clinical data; creating a similar patient cohort by aggregating data from the identified similar patients, where the aggregated data includes demographic data, clinical data and medical images of the similar patients; retrieving the medical images from the similar patient cohort; performing radiomics-derived quantitative analysis on the retrieved medical images to train an ANN classification model; applying the lesion to the trained ANN classification model to predict histological nature of the lesion; and determining diagnosis and medical treatment of the patient based on the predicted histological nature.

### BACKGROUND

The practice of medicine generally involves prevention, diagnosis, mitigation and treatment of disease. The variety and complexity of human morbidities make it effectively impossible to always reach the most appropriate diagnosis and treatment, or to predict with certainty a particular response to treatment. Where multi-pronged treatment is known to increase the chances of successfully treating certain metastatic cancers, reduction in treatment morbidity is key to survivorship.

Critical surgical procedures to remove residual metastatic lesions (e.g., tumors) post chemotherapy often come with short- and long-term complications, such as time off from work, aesthetic risks, vascular complications, scarring and organ damage. Existing clinical image based predictive algorithms are not sufficiently sensitive and cannot discriminate lesions in enough detail to become the basis of medical/surgical decision-making.

Radiologists are tasked with viewing medical images, and generating radiology reports describing images, including computed tomography (CT) images, X-ray images, magnetic resonance imaging (MRI) images, positron emission tomography (PET) scans, and ultrasound images. The diagnosis and treatment determinations, as well as predictions of successful outcomes, often depend on the information provided by these medical images and associated radiology reports. Standard imaging though may not reliably differentiate between benign and potentially invasive growths or treatment response. Baseline clinical and histopathologic options have been tested for detecting factors indicative of response to various treatments (e.g., radiation, chemotherapy). The baseline options may include interval size reduction of lesions in response to various treatments, pre- and post-chemo tumor marker levels, nodal size, primary tumor activity and detection of residual active disease, for example, all of which are indicators of survivorship and well-being. There is need for image based predictive algorithms sensitive enough to base important treatment decisions on.

Radiomics is an emerging field that converts medical imaging data, for example, to quantitative biomarkers by application of advanced computational methodologies. Such computational methodologies may include quantitative imaging features (e.g., extracted from a defined tumor region of interest in a scan and including descriptors of intensity distribution), texture heterogeneity patterns, and spatial relationships between the various intensity levels not detectable by the human eye. A large set of features can be subsequently tested for accuracy in predicting treatment outcomes, even when physiologic underpinnings are unknown.

Radiomics generally uses image processing techniques to extract quantitative features from regions of interest to train a machine intelligence classifier that predicts outcomes. CT-derived tumor textural heterogeneity and PET-derived textural heterogeneity, for example, are considered independent predictors of survival. In a radiomics analysis of features extracted from CT data of patients with lung or head-and-neck cancer, for example, a large number of radiomics features were proven to have diagnostic and prognostic power. However, broad application of radiomics does not provide sufficiently detailed or relevant information to provide the predictive certainty desired for treating patients according to their unique demographic and medical circumstances.

There is need for algorithms sensitive enough on which to base important diagnostic and treatment decisions using highly relevant information derived from medical images of similar patients. Radiomics is a powerful tool toward achieving this end, but the conventional use of radiomics falls short, and is overly dependent on user skill and interaction.

### SUMMARY

According to a representative embodiment, a method is provided for predicting histopathological nature of lesions of a patient. The method includes detecting at least one lesion in a medical image of the patient; extracting image findings from a radiology report describing the medical image, including the at least one lesion, using a natural language processing (NLP) algorithm; retrieving demographic and clinical data of the patient from at least one of a picture archiving and communication system (PACS) or a radiology information system (RIS); identifying multiple similar patients based on the extracted image findings and the demographic and clinical data of the patient; creating a similar patient cohort by aggregating data from the identified similar patients, where the aggregated data includes demographic data, clinical data and medical images of the similar patients, respectively; retrieving the medical images from the similar patient cohort; performing radiomics-derived quantitative analysis on the retrieved medical images to train an artificial neural network (ANN) classification model; and applying the at least one lesion to the trained ANN classification model to predict a histological nature (including malignancy where applicable) of the at least one lesion. The diagnosis and/or medical management of the patient for the at least one lesion may be based on the predicted histological nature of the at least one lesion.

According to another representative embodiment, a system is provided for predicting histopathological nature of lesions of a patient. The system includes at least one processor; at least one database storing demographic data, clinical data and medical images of a plurality of patients; a graphical user interface (GUI) enabling a user to interface with the processor; and a non-transitory memory storing instructions that, when executed by the processor, cause the at least one processor to: detect at least one lesion in a medical image of the patient; extract image findings from a radiology report describing the medical image, including the at least one lesion, using an NLP algorithm; retrieve demographic and clinical data of the patient from the at least one database; identify similar patients from among the plurality of patients by searching the at least one database based on the extracted image findings and the demographic and clinical data of the patient; create a similar patient cohort by aggregating data from the identified similar patients, where the aggregated data includes demographic data, clinical data and medical images of the similar patients, respectively; retrieve the medical images from the similar patient cohort; perform radiomics-derived quantitative analysis on the retrieved medical images to train an ANN classification model; apply the at least one lesion to the trained ANN classification model to predict a histological nature of the at least one lesion; and display the predicted histological nature of the at least one lesion on the GUI. The medical diagnosis and/or medical management of the patient for the at least one lesion may be determined based on the predicted histological nature of the at least one lesion.

According to another representative embodiment, a non-transitory computer readable medium is provided that stores instructions for predicting histopathological nature of lesions of a patient that, when executed by one or more processors, cause the one or more processors to detect at least one lesion in a medical image of the patient; extract image findings from a radiology report describing the medical image, including the at least one lesion, using an NLP algorithm; retrieve demographic and clinical data of the patient from the at least one database; identify similar patients from among the plurality of patients by searching the at least one database based on the extracted image findings and the demographic and clinical data of the patient; create a similar patient cohort by aggregating data from the identified similar patients, wherein the aggregated data includes demographic data, clinical data and medical images of the similar patients, respectively; retrieve the medical images from the similar patient cohort; perform radiomics-derived quantitative analysis on the retrieved medical images to train an ANN classification model; apply the at least one lesion to the trained ANN classification model to predict a histological nature of the at least one lesion; and display the predicted histological nature of the at least one lesion. The medical diagnosis and/or medical management of the patient for the at least one lesion may be determined based on the predicted histological nature of the at least one lesion.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for predicting the histopathological nature of lesions of a patient, according to a representative embodiment.

FIG. 2 is a flow diagram showing a method of predicting the histopathological nature of lesions of a patient, according to a representative embodiment.

FIG. 3 is a flow diagram of a method of performing radiomics-derived quantitative analysis, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein provide an automated system for applying the power of high discriminative accuracy of quantitative radiomics to personalized care of individual patients. The embodiments enable aggregate data of similar patient cohorts built from electronic health record data using radiomics to be leveraged to make critical and lifesaving treatment optimization that may have a direct effect on survivorship, as well as clinical treatment timeliness and quality.

Generally, radiomics is a known quantitative approach to medical imaging, which aims at enhancing existing data available to clinicians by means of advanced mathematical analysis. Radiomics assumes that medical images contain information of disease-specific processes that are imperceptible by the human eye, and thus are not accessible through traditional visual inspection of the generated images. Through mathematical extraction of spatial distribution of signal intensities and pixel interrelationships, radiomics is able to quantify textural information from the medical images using known analysis methods from the field of artificial intelligence. Visually appreciable differences in image intensity, shape and/or texture may be quantified using radiomics, which helps to remove user subjectivity from image interpretation. Recently, radiomics has been applied in the field of oncology, and provides additional data for diagnosis and medical management, including determining medical treatment, determinations. Radiomics analysis may be accomplished in a variety of medical images from different modalities, with the potential for additive value of extracted imaging information integrated across modalities.

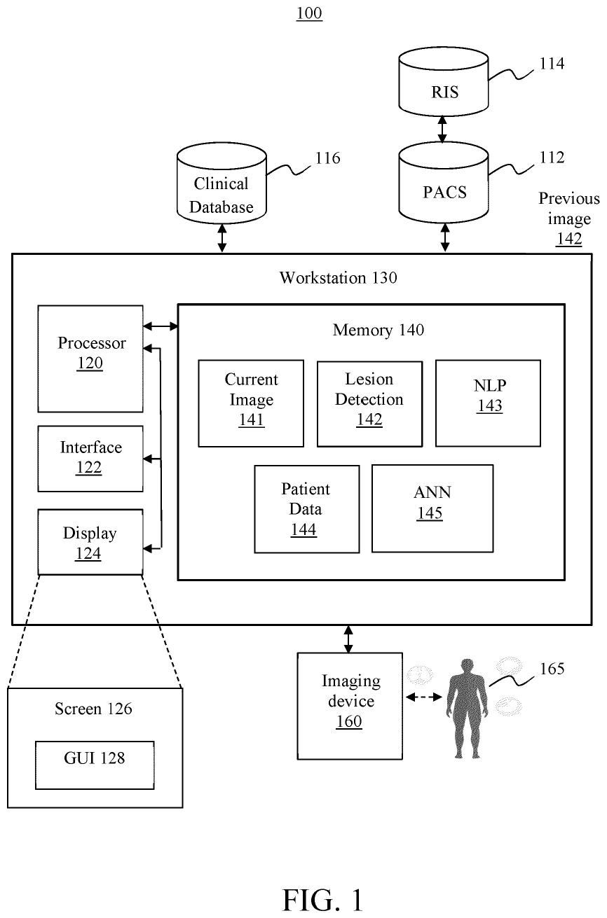

FIG. 1 is a simplified block diagram of a system for predicting the histopathological nature of lesions in an image of a patient, in order to determine and provide diagnosis and medical treatment, using information derived from similar patients as a guide, according to a representative embodiment.

Referring to FIG. 1, system includes a workstation 130 for implementing and/or managing the processes described herein. The workstation 130 includes one or more processors indicated by processor 120, one or more memories indicated by memory 140, interface 122 and display 124. The processor 120 may interface with an imaging device 160 through an imaging interface (not shown). The imaging device 160 may be any of various types of medical imaging device/modality, including an X-ray imaging device, a CT scan device, an MRI device, a PET scan device, or an ultrasound imaging device, for example.

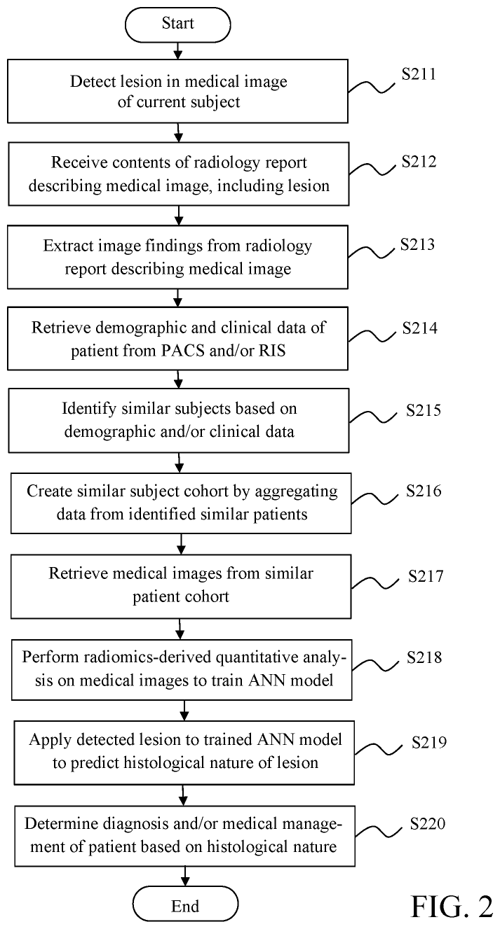

The memory 140 stores instructions executable by the processor 120. When executed, the instructions cause the processor 120 to implement one or more processes for predicting the nature of the lesions using medical images of similar patients, described below with reference to FIG. 2, for example. For purposes of illustration, the memory 140 is shown to include software modules, each of which includes the instructions corresponding to an associated capability of the system 100, as discussed below.

The processor 120 is representative of one or more processing devices, and may be implemented by field programmable gate arrays (FPGAs), application specific integrated circuits (ASICs), a digital signal processor (DSP), a general purpose computer, a central processing unit, a computer processor, a microprocessor, a microcontroller, a state machine, programmable logic device, or combinations thereof, using any combination of hardware, software, firmware, hard-wired logic circuits, or combinations thereof. Any processing unit or processor herein may include multiple processors, parallel processors, or both. Multiple processors may be included in, or coupled to, a single device or multiple devices. The term “processor” as used herein encompasses an electronic component able to execute a program or machine executable instruction. A processor may also refer to a collection of processors within a single computer system or distributed among multiple computer systems, such as in a cloud-based or other multi-site application. Programs have software instructions performed by one or multiple processors that may be within the same computing device or which may be distributed across multiple computing devices.

The memory 140 may include main memory and/or static memory, where such memories may communicate with each other and the processor 120 via one or more buses. The memory 140 may be implemented by any number, type and combination of random access memory (RAM) and read-only memory (ROM), for example, and may store various types of information, such as software algorithms, artificial intelligence (AI) machine learning models, and computer programs, all of which are executable by the processor 120. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 140 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 140 may store software instructions and/or computer readable code that enable performance of various functions. The memory 140 may be secure and/or encrypted, or unsecure and/or unencrypted.

The system 100 also includes databases for storing information that may be used by the various software modules of the memory 140, including a picture archiving and communication systems (PACS) database 112, a radiology information system (RIS) database 114, and clinical database 116. The clinical database generally refers to locations where patients' clinical information can be found. Examples of clinical databases include electronic medical records (EMR) databases, data warehouses, data repositories, and the like. The PACS database 112, the RIS database 114 and the clinical database 116 may be implemented by any number, type and combination of RAM and ROM, for example. The various types of ROM and RAM may include any number, type and combination of computer readable storage media, such as a disk drive, flash memory, EPROM, EEPROM, registers, a hard disk, a removable disk, tape, CD-ROM, DVD, floppy disk, Blu-ray disk, USB drive, or any other form of storage medium known in the art. The databases are tangible storage mediums for storing data and executable software instructions and are non-transitory during the time data and software instructions are stored therein. The databases may be secure and/or encrypted, or unsecure and/or unencrypted. For purposes of illustration, the PACS database 112, the RIS database 114 and the clinical database 116 are shown as separate databases, although it is understood that they may be combined, and/or included in the memory 140, without departing from the scope of the present teachings. The clinical database 116 may be built as a matter of routine at one or more facilities providing clinical care, storing at least patient demographic and clinical information.

The processor 120 may include or have access to an AI engine or module, which may be implemented as software that provides artificial intelligence, such as natural language processing (NLP) algorithms, and applies machine learning, such as artificial neural network (ANN) modeling, described herein. The AI engine may reside in any of various components in addition to or other than the processor 120, such as the memory 140, an external server, and/or the cloud, for example. When the AI engine is implemented in a cloud, such as at a data center, for example, the AI engine may be connected to the processor 120 via the internet using one or more wired and/or wireless connection(s).

The interface 122 may include a user and/or network interface for providing information and data output by the processor 120 and/or the memory 140 to the user and/or for receiving information and data input by the user. That is, the interface 122 enables the user to enter data and to control or manipulate aspects of the processes described herein, and also enables the processor 120 to indicate the effects of the user's control or manipulation. All or a portion of the interface 122 may be implemented by a graphical user interface (GUI), such as GUI 128 viewable on the display 124, discussed below. The interface 122 may include one or more of ports, disk drives, wireless antennas, or other types of receiver circuitry. The interface 122 may further connect one or more user interfaces, such as a mouse, a keyboard, a trackball, a joystick, a microphone, a video camera, a touchpad, a touchscreen, voice or gesture recognition captured by a microphone or video camera, for example.

The display 124, also referred to as a diagnostic viewer, may be a monitor such as a computer monitor, a television, a liquid crystal display (LCD), an organic light emitting diode (OLED), a flat panel display, a solid-state display, or a cathode ray tube (CRT) display, or an electronic whiteboard, for example. The display 124 includes a screen 126 for viewing internal images of a current subject (patient) 165, as well as the GUI 128 to enable the user to interact with the displayed images and features.

Referring to the memory 140, current image module 141 is configured to receive (and process) a current medical image corresponding to the current patient 165 for display on the display 124. The current medical image is the image currently being read/interpreted by the user (e.g., radiologist) during a reading workflow. The current medical image may be received from the imaging device 160, for example, during a contemporaneous imaging session of the patient. Alternatively, the current image module 141 may retrieve the current medical image from the PACS database 112, which has been stored from the imaging session, but not yet read by the user. The current medical image is displayed on the screen 126 to enable analysis by the user for preparing a radiology report, discussed below.

Lesion detection module 142 detects abnormalities in the current medical image of the current patient 165, including lesions which may be cancerous. The lesion detection module 142 may detect lesions automatically using well known image segmentation techniques, such as U-Net, for example. Alternatively, lesion detection module 142 may detect lesions interactively, where the user selects margins of an apparent lesion or designates a region of interest of the current medical image using the interface 122 and the GUI 128. The lesion detection module 142 fills in an area within the selected margins, or performs segmentation only within the designated region of interest. In alternative embodiments, the lesions are detected manually by the user via the interface 122 and the GUI 128 without segmentation.

The user prepares (e.g., dictates) the radiology report using interface 122 and the GUI 128. The radiology report includes measurements and corresponding descriptive text of the lesions in the current medical image detected by the lesion detection module 142. The measurements and descriptive text may be included in the findings section and/or the impressions section of the radiology report, for example. Generally, the findings section of the radiology report includes observations by the user about the medical images, and the impressions section includes conclusions and diagnoses of medical conditions or ailments determined by the user, as well as recommendations regarding follow-up medical management, such as medical treatment, testing, additional imaging and the like, for example. The radiology report is stored in the PACS database 112, the RIS database 114, and/or the clinical database 116.

NLP module 143 is configured to execute one or more NLP algorithms using word embedding technology to extract image findings from the contents of the radiology report by processing and analyzing natural language data. That is, the NLP module 143 evaluates the sentences in the radiology report, and extracts measurements of the lesions observed in the current image, as well as descriptive text associated with the measurements as entered by the user. The descriptive text may include information such as temporality of a measurement, a series number of the image for which the measurement is reported, an image number of the image for which the measurement is reported, an anatomical entity in which the associated abnormality is found, a RadLex® description of the status of the lesion or other observation, an imaging description of the area being imaged, and a segment number of the organ being imaged, for example. NLP is well known, and may include syntax and semantic analyses, for example, and deep learning for improving understanding by the NLP module 143 with the accumulation of data, as would be apparent to one skilled in the art. The extracted image findings are stored in the PACS database 112, the RIS database 114, and/or the clinical database 116.

A patient data module 144 is configured to retrieve demographic and clinical data of the patient from one or more databases, such as the PACS database 112, the RIS database 114 and/or the clinical database 116. The demographic data provides characteristics of the patient, such as age, race, gender, height, weight, and the like. The clinical data provides medical history and condition of the patient, such as allergies, medications, symptoms, labs, current and previous medical diagnoses, current and previous medical conditions, and current and previous medical treatments.

In addition, the patient data module 144 is configured to identify patients who are in similar circumstances as the current patient using the extracted image findings, and the retrieved demographic and clinical data of the current patient. In particular, the patient data module 144 searches one or more databases, such as the clinical database 116, containing image findings, demographic data and clinical data of a population of patients. For example, the patient data module 144 may build a query with search terms indicative of all or part of the image findings, the demographic data and the clinical data of the current patient. The patient data module 144 then searches the clinical database 116 using the query, and determines matches between the search terms and the image findings, demographic data and clinical data of the patients. The patients whose data matches a predetermined number or percentage of the query search terms are identified as similar patients.

The patient data module 144 then retrieves similar patient data, which includes the medical images and the demographic and clinical data associated with the patients that have been identified as similar patients. The medical images may be retrieved from one or more image databases, such as the PACS database 112 and/or the RIS database 114, and the demographic and clinical data may be retrieved from the clinical database 116, for example. The patient data module 144 creates a similar patient cohort by aggregating the retrieved similar patient data of the similar patients, including the medical images, the demographic data, and the clinical data of the similar patients. The aggregated data may be stored temporarily in a cloud storage device, for example.

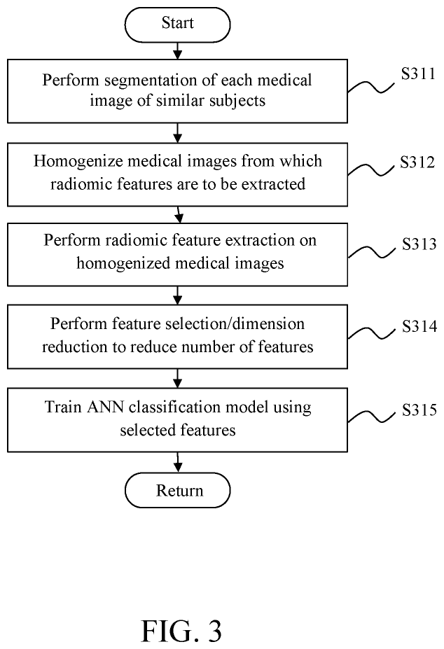

An ANN module (for image classification) 145 is configured to train an ANN classification model using the retrieved medical images from the patient cohort so that the ANN classification model is tailored to the current patient 165, and to apply the current medical image of the current patient 165 to the ANN classification model in order to predict a histological nature of each lesion detected in the medical image. That is, the ANN module 145 performs radiomics-derived quantitative analysis on the retrieved medical images from the patient cohort to provided selected features, as discussed below with reference to FIG. 3. The ANN classification model is trained using the selected features provided by the radiomics-derived quantitative analysis.

The results of the applying the current medical image to the ANN classification model are displayed on the display 124. Based on the displayed results, the user is able to accurately diagnose the histological nature and phenotype of the lesion(s) in the current medical image, and to determine the best medical management of the current patient 165 (e.g., radiation, chemotherapy, resection) and the likely outcome of such treatment in view of the similar patient cohort data. The medical management is then implemented, and may be tracked so that the results for the current patient 165 may be added to the clinical database 116 for use with regard to future patients.

In various embodiments, all or part of the processes provided by the NLP module 143 and/or the ANN module 145 may be implemented by an AI engine, for example.

FIG. 2 is a flow diagram of a method of predicting histopathological nature of lesions of a patient, according to a representative embodiment. The method may be implemented by the system 100, discussed above, under control of the processor 120 executing instructions stored as the various software modules in the memory 140, for example.

Referring to FIG. 2, a lesion is detected in a medical image of a current patient in block S211. The medical image may be obtained and displayed during a current imaging exam for a particular study of the current patient. Of course, multiple lesions may be detected in the same medical image, in which case the steps described herein would be applied to each of the detected lesions. The medical image may be received directly from a medical imaging device/modality (e.g., imaging device 160), such as an X-ray imaging device, a CT scan device, an MR imaging device, a PET scan device or an ultrasound imaging device, for example. Alternatively, or in addition, the medical image may be retrieved from a database (e.g., PACS database 112, RIS database 114), for example, in which the medical image has been previously stored following a current imaging exam. The medical image may be displayed on any compatible display (e.g., display 124), such as a diagnostic viewer, routinely used for reading radiological studies.

The lesion may be detected automatically using well known image segmentation techniques, such as U-Net, for example. Alternatively, the lesion may be detected on the display interactively by a user (e.g., radiologist) using a GUI. For example, the user may use a mouse or other user interface to select margins of an apparent lesion or region of interest containing the lesion. The interior portion of the lesion may then be filled in or otherwise highlighted automatically, or the image segmentation may be performed only in the selected region of interest.

In block S212, contents of a radiology report are received from the user via the GUI describing the medical image of the current patient, including the lesion. The contents of the radiology report include image findings, which provide measurements of the lesion and descriptive text associated with the measurements. The radiology report may be dictated, for example, by the user viewing the displayed medical image.

In block S213, the image findings are extracted from the contents of the radiology report describing the medical image, including the description of the lesion. The image findings may be extracted using a known NLP algorithm, for example. Generally, the NLP algorithm parses the measurements and the descriptive text in the radiology report to identify numbers, key words and key phrases indicative of the at least one lesion. The NLP extraction may be performed automatically, without explicit inputs from the user who is reviewing the medical image. Relevant data from the radiology report contents may be extracted by applying domain-specific contextual embeddings for successful extraction of the measurements and descriptive text of the lesion. The NLP extraction may take place at the time the radiology report is created, for example.

In block S214, demographic and clinical data of the current patient are retrieved from a patient database, such as a PACS and/or a RIS, for example. The demographic and clinical data, together with the extracted image findings, provide a comprehensive portrayal of the current patient and the current state of their condition.

In block S215, patients having similar demographic and clinical circumstances to those of the current patient are identified based on the image findings and the demographic and clinical data of the current patient. The similar patients may be identified by searching a clinical database of patients (e.g., clinical database 116), which includes previously obtained image findings, demographic data and/or clinical data of past patients. The clinical database may be built as a matter of routine at one or more facilities providing clinical care. In fact, the current patient's image findings and demographic and clinical data may be added to the clinical database for use in analyzing the conditions of subsequent patients. Relevant image findings, demographic data and/or clinical data of the similar patients may be identified using a query containing search terms indicative of the image findings, demographic data and/or clinical data of the current patient.

Determining whether the patients have similar circumstances may be accomplished in various ways, without departing from the scope of the present teachings. For example, the clinical database may be searched using a query containing a number of search terms that describe the circumstances of the current patient, including the image findings, the demographic data and the clinical data (e.g., symptoms, diagnoses, medications, labs) of the current patient. An illustrative example of a query may be “50-year-old, African American, female, diabetic, with breathlessness, chronic obstructive pulmonary disease (COPD), on Glipizide/Metformin with ‘nodule’ on CT chest.” Then, patients in the clinical database whose data matches a predetermined number or percentage of the query search terms may be identified as similar patients.

In block S216, a similar patient cohort is created by aggregating data from the similar patients identified in block S215. In addition to the similar patients' demographic and clinical data retrieved from the clinical database, the aggregated data further includes medical images of the similar patients. The similar patients' medical images are stored in association with the respective demographic and clinical data. For example, the medical images may be stored in the clinical database with the demographic and clinical data, or the clinical database may be updated to reference the medical images already stored in a separate imaging database, such as the PACS database or the RIS database, for example.

In block S217, the medical images of the similar patients in the similar patient cohort are retrieved from the database(s) in which they have been stored. Radiomics-derived quantitative analysis is then performed in block S218 on the retrieved medical images in order to train an ANN classification model based on the similar patient cohort.

FIG. 3 is a flow diagram of a method of performing radiomics-derived quantitative analysis, indicated in block S218 of FIG. 2, according to a representative embodiment. The method may be implemented by the system 100, discussed above, under control of the processor 120 executing instructions stored as the various software modules in the memory 140, such as ANN module 145, for example.

Referring to FIG. 3, the radiomics-derived quantitative analysis begins in block S311 by performing segmentation of each of the medical images (matched by modality with current patient) of the similar patients retrieved in block S217. To perform segmentation, a region of interest (ROI) or volume of interest (VOI) is demarked in each of the medical images, where ROIs apply to two-dimensional images and VOIs apply to three-dimensional images. The segmentation is performed automatically in each of the ROIs and VOIs to identify the respective lesions, thus avoiding user variability of radiomic features.

In block S312, the medical images from which radiomic features are to be extracted are homogenized with respect to pixel spacing, grey-level intensities, bins of the grey-level histogram, and the like. The ROIs and VOIs associated with the lesions are delineated, e.g., using an ITK-SNAP application. The delineated ROIs and VOIs are interpolated by applying any compatible interpolation algorithm, such as trilinear interpolation, tricubic convolution and tricubic spline interpolation, for example. The interpolation enables texture feature sets to become rotationally invariant to allow comparison between image data from different samples, cohorts and batches, and to increase reproducibility between different datasets. Range re-segmentation and intensity outlier filtering (normalization) are performed to remove pixels/voxels from the segmented regions that fall outside of a specified range of grey-levels. Discretization of image intensities inside the ROIs or VOIs is preformed by grouping the original values according to specific range intervals (bins). The homogenization of the medical images is conceptually equivalent to creating o a histogram.

In block S313, radiomic feature extraction (calculation) is performed on the homogenized medical images. Feature descriptors corresponding to extracted features are used to quantify characteristics of the grey levels within the ROIs or VOIs. Image Biomarker Standardization Initiative (IBSI) guidelines, for example, provide standardized feature calculations. Different types (i.e., matrices) of radiomic features exist, such as intensity (histogram)-based features, shape features, texture features, transform-based features, and radial features, for example. Numerous radiomic features may be extracted from the medical images, including size and shape based-features, descriptors of image intensity histograms, descriptors of relationships between image pixels/voxels, textures extracted from wavelet and Laplacian of Gaussian filtered images, and fractal features, for example. The descriptors of the relationships between image pixels/voxels may include gray-level co-occurrence matrix (GLCM), run length matrix (RLM), size zone matrix (SZM), and neighborhood gray tone difference matrix (NGTDM) derived textures, for example.

In block S314, feature selection and dimension reduction are performed in order to reduce the number of features to be used for building the ANN classification model for the similar patient cohort, thereby generating valid and generalized results. Performing feature selection and dimension reduction includes excluding all non-reproducible features, selecting most relevant variables for various tasks (e.g., machine learning techniques like knock-off filters, recursive feature elimination methods, random forest algorithms), building correlation clusters of highly correlated features in the data and allowing selection of only one representative feature per correlation cluster, selecting a most representative variation of the variations within the similar patient cohort, and performing data visualization to see dimensionality reduction. Accordingly, performance of the feature selection and dimension reduction provides non-correlated, highly relevant features.

In block S315, the ANN classification model is trained using the features selected in block S314 for performing the classification tasks. This includes splitting the selected features into a training and testing dataset and a validation dataset. Using these datasets, the ANN classification model differentiates the lesion in each of the medical images as being malignant versus benign, for example, and trains the ANN classification model accordingly.

Radiomics presents high-throughput extraction of advanced quantitative features to describe tumor phenotypes objectively and quantitatively. Radiomic textural features describe distinctive tumor phenotype (appearance) that may be driven by underlying genetic and biological variability.

Referring again to FIG. 2, in block S219, the lesion in the current patient detected in block S212 is applied to the trained ANN classification model to predict the histological nature of the lesion. Since the ANN classification model has been trained using the similar patient cohort specific to the current patient, as well as radiomics-derived quantitative analysis to detect features not otherwise identifiable by the user simply viewing the medical images on a display, the predicted histological nature of the lesion will be significantly more accurate and clinically relevant than one predicted using a more generalized training regimen. Also, performing radiomics-derived quantitative analysis on the retrieved medical images to training the ANN classification model using radiomics-derived quantitative analysis; and the lesion to the trained ANN classification model to predict a histological nature of the lesion are not concepts that can be performed in the human mind.

In block S220, diagnosis and medical management of the patient with regard to the lesion is determined based on the predicted histological nature of the lesion. That is, the predicted histological nature of the lesion provides direction to the user (clinician) as to the nature of the lesion, and likely responses to various treatment choices, such as radiation, chemotherapy or resection. For example, the user specifically needs to know whether the residual disease of the lesion is likely malignant and has potential to spread and grow, or just fibrosis that does not need surgical resection, thus avoiding accompanying short and long term surgical complications. The appropriate medical management of the patient is then implemented, and tracked so that results may be added to the clinical database for use with regard to future patients.

Using the results of applying the lesion to the trained ANN classification model in order to diagnose the lesion and to determine and implement appropriate medical management, the user will be able to gauge whether the lesion has responded or will respond adequately to treatment, and whether the histopathological picture has changed over time. Quantitative radiomics applied to the medical images of the current patient and to those of the similar patient cohort provides evidence and strengthens the prediction of histopathological picture to differentiate benign from malignant disease, and to add to the certainty of response to treatment and to optimize future therapy. Applying radiomics features to the similar patient cohort in this manner is a highly accurate, non-invasive, time effective way to help guide crucial treatment decisions.

Therefore, according to various embodiments, a similar patient cohort is developed by searching a database of patients using extracted image findings and the demographic and clinical data of a current patient, and a model is created and trained on radiomics-derived quantitative analysis of medical images associated with the similar patients. The user therefore draws very relevant insights from the similar patient cohort that the user would be unable to determine by reviewing the medical images of the similar patient cohort merely using the naked eye, and applies them to the current patient. The radiomics analysis of features extracted from the medical images enables accurate and clinically relevant prediction of treatment response, differentiation of benign and malignant tumors, delineation primary and nodal tumors from normal tissues, and assessment of cancer genetics in many cancer types, for example. Intra-lesion heterogeneity and inter-lesion heterogeneity, for example, provide valuable information for personalized therapy by directing treatment planning.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although diagnosing and determining medical management for lesions of a patient have been described with reference to exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Also, although diagnosing and determining medical management for lesions has been described with reference to particular means, materials and embodiments, there is no intention to be limited to the particulars disclosed; rather the embodiments extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of predicting histopathology of lesions of a patient, the method comprising:
detecting at least one lesion in a medical image of the patient;
extracting image findings from a radiology report describing the medical image, including the at least one lesion, using a natural language processing (NLP) algorithm;
retrieving demographic and clinical data of the patient from at least one of a picture archiving and communication system (PACS) or a radiology information system (RIS);
identifying a plurality of similar patients based on the extracted image findings and the demographic and clinical data of the patient;
creating a similar patient cohort by aggregating data from the identified plurality of similar patients, wherein the aggregated data includes demographic data, clinical data and medical images of the similar patients, respectively;
retrieving the medical images from the similar patient cohort;
performing radiomics-derived quantitative analysis on the retrieved medical images to train an artificial neural network (ANN) classification model; and
applying the at least one lesion to the trained ANN classification model to predict a histological nature of the at least one lesion.

2. The method of claim 1, further comprising:
determining medical diagnosis and medical treatment of the patient for the at least one lesion based on the predicted histological nature of the at least one lesion.

3. The method of claim 1, wherein the at least one lesion is detected by image segmentation.

4. The method of claim 1, wherein identifying the plurality of similar patients comprises:
searching a clinical database of patients using a query having search terms indicative of the demographic and clinical data of the patient; and
identifying patients in the clinical database matching a predetermined number or percentage of the search terms as similar patients.

5. The method of claim 3, wherein the clinical database comprises at least one of electronic medical records (EMR) database, a clinical data warehouse, or a data repository.

6. The method of claim 1, wherein extracting the image findings from the radiology report using the NLP algorithm comprises applying domain-specific contextual embeddings.

7. The method of claim 1, wherein the demographic and clinical data comprise at least age, gender and race of the patient, and past and current medical diagnoses and treatments.

8. The method of claim 1, wherein the demographic and clinical data are stored in a clinical database, and the medical images of the similar patients are stored in an imaging database separate from the clinical database, and wherein the clinical database is updated to reference the medical images in the separate imaging database.

9. The method of claim 1, wherein the demographic and clinical data are stored in a clinical database, and the medical images of the similar patients are stored in the clinical database in association with the demographic and clinical data.

10. The method of claim 1, wherein performing the radiomics-derived quantitative analysis on the retrieved medical images to train the ANN classification model comprises:
performing segmentation of the medical images of the similar patients;
homogenizing the medical images with respect to one or more of pixel spacing, grey-level intensities, and bins of a grey-level histogram;
performing radiomic feature extraction on the homogenized medical images; and
performing feature selection and dimension reduction to reduce features to be used for training the ANN classification model for the similar patient cohort.

11. The method of claim 1, wherein applying the at least one lesion to the trained ANN classification model to predict the histological nature of the at least one lesion comprises predicting malignancy of the at least one lesion.

12. A system for predicting histological nature for lesions of a patient, the system comprising:
at least one processor;
at least one database storing demographic and clinical data and medical images of a plurality of patients;
a graphical user interface (GUI) enabling a user to interface with the processor; and
a non-transitory memory storing instructions that, when executed by the processor, cause the at least one processor to:
detect at least one lesion in a medical image of the patient;
extract image findings from a radiology report describing the medical image, including the at least one lesion, using a natural language processing (NLP) algorithm;
retrieve demographic and clinical data of the patient from the at least one database;
identify similar patients from among the plurality of patients by searching the at least one database based on the extracted image findings and the demographic and clinical data of the patient;
create a similar patient cohort by aggregating data from the identified similar patients, wherein the aggregated data includes demographic and clinical data and medical images of the similar patients, respectively;
retrieve the medical images from the similar patient cohort;
perform radiomics-derived quantitative analysis on the retrieved medical images to train an artificial neural network (ANN) classification model;
apply the at least one lesion to the trained ANN classification model to predict a histological nature of the at least one lesion; and
display the predicted histological nature of the at least one lesion on the GUI,
wherein medical diagnosis and/or medical treatment of the patient for the at least one lesion is determined based on the predicted histological nature of the at least one lesion.

13. The system of claim 12, wherein the at least one lesion is detected by image segmentation.

14. The system of claim 12, wherein the instructions cause the at least one processor to identify the similar patients by:
searching the at least one database using a query having search terms indicative of the demographic and clinical data of the patient; and
identifying patients in the at least one database matching a predetermined number or percentage of the search terms as similar patients.

15. The system of claim 14, wherein the at least one database comprises at least one of electronic medical records (EMR) database, a clinical data warehouse, or a data repository.

16. The system of claim 12, wherein the demographic and clinical data comprise at least age, gender and race of the patient, and past and current medical diagnoses and treatments.

17. The system of claim 12, wherein the instructions cause the at least one processor to perform the radiomics-derived quantitative analysis on the retrieved medical images to train the ANN classification model by:
performing segmentation of the medical images of the similar patients;
homogenizing the medical images with respect to one or more of pixel spacing, grey-level intensities, and bins of a grey-level histogram;
performing radiomic feature extraction on the homogenized medical images; and
performing feature selection and dimension reduction to reduce features to be used for training the ANN classification model for the similar patient cohort.

18. The system of claim 12, wherein predicting the histological nature of the at least one lesion comprises predicting malignancy of the at least one lesion.

19. A non-transitory computer readable medium storing instructions for predicting histological nature of lesions of a patient that, when executed by one or more processors, cause the one or more processors to:
detect at least one lesion in a medical image of the patient;
extract image findings from a radiology report describing the medical image, including the at least one lesion, using a natural language processing (NLP) algorithm;
retrieve demographic and clinical data of the patient from at least one database;
identify similar patients from among a plurality of patients by searching the at least one database based on the extracted image findings and the demographic and clinical data of the patient;
create a similar patient cohort by aggregating data from the identified similar patients, wherein the aggregated data includes demographic and clinical data and medical images of the similar patients, respectively;
retrieve the medical images from the similar patient cohort;
perform radiomics-derived quantitative analysis on the retrieved medical images to train an artificial neural network (ANN) classification model;
apply the at least one lesion to the trained ANN classification model to predict a histological nature of the at least one lesion; and
display the predicted histological nature of the at least one lesion,
wherein medical diagnosis and/or medical treatment of the patient for the at least one lesion is determined based on the predicted histological nature of the at least one lesion.

20. The non-transitory computer readable medium of claim 18, wherein the instructions cause the one or more processors to identify the similar patients by:
searching the at least one database using a query having search terms indicative of the demographic and clinical data of the patient; and
identifying patients in the at least one database matching a predetermined number or percentage of the search terms as similar patients.
